(19) United States
(12) Patent Application Publication
(10) Pub. No.: US 2014/0336488 A1
(43) Pub. Date: Nov. 13, 2014

(54) DEVICES AND METHODS FOR THE RAPID AND ACCURATE DIAGNOSIS AND TREATMENT OF SINUSITIS

(71) Applicant: Subinoy Das, Dublin, OH (US)

(72) Inventor: Subinoy Das, Dublin, OH (US)

(73) Assignee: The Ohio State University, Columbus, OH (US)

(21) Appl. No.: 14/343,561

(22) PCT Filed: Sep. 14, 2012
(86) PCT No.: PCT/US12/55401

§ 371 Date: Jul. 28, 2014

Related U.S. Application Data
(60) Provisional application No. 61/534,577, filed on Sep. 14, 2011.

(51) Int. Cl. A61B 5/145; A61B 5/00

(52) U.S. Cl. CPC A61B 5/14507; A61B 5/6847
USPC 600/367; 600/309

(57) ABSTRACT

Disclosed are probes, systems, and devices that can be used to assay the presence of a biomarker in the sinus of a patient. The probes, systems, and devices can be used to contact the sinus fluid inside the sinus of a patient with an immunoassay for the presence of a biomarker that is characteristic of sinusitis. The probes can therefore be used to determine the underlying cause of sinusitis in a patient. Also provided are methods of treating sinusitis by administering to a patient a therapeutic regimen selected in view of a biomarker detected in the sinus of the patient. As the biomarker is associated with one or more causative agents of sinusitis, a therapy effective to treat the underlying causative agent can be selected.

### CROSS REFERENCE TO RELATED APPLICATIONS

This application claims the benefit of U.S. Provisional Application No. 61/534,577, filed Sep. 14, 2011, which is incorporated herein by reference in its entirety.

### STATEMENT REGARDING FEDERALLY SPONSORED RESEARCH OR DEVELOPMENT

This invention was made with Government Support under Agreements NIH/NIDCD R01DC05847-0651 and NIH/OD KL2RR025754 awarded to Subinoy Das by the National Institutes of Health. The Government has certain rights to the invention.

### FIELD

The present disclosure is generally related to medical devices for the rapid and accurate diagnosis of sinusitis, as well as methods of diagnosing and treating sinusitis.

### BACKGROUND

The bones of the nose and face contain a series of cavities known as paranasal sinuses. The paranasal sinuses, which include the frontal sinuses, ethmoid sinuses, sphenoidal sinuses, and maxillary sinuses, are connected to the nasal cavity via a series of passageways. The sinuses serve to protect the lungs by moistening and warming inspired air.

The paranasal sinuses are lined with mucous-producing epithelial tissue. In healthy persons, the mucus produced by the lining of the paranasal sinuses drains from each sinus cavity into the nasopharynx via an opening known as an ostium. When the ostium or regions near the ostium become inflamed, the egress of mucus from the sinus is interrupted. Occlusion of an ostium can lead to an inflammation or infection of the paranasal sinus, termed rhinosinusitis or sinusitis. Symptoms of sinusitis can include headaches, facial pain or pressure, nasal congestion or post-nasal drainage, pain in the upper teeth, bad breath, difficulty breathing through one or both nostrils, diminished sense of taste and/or smell, cough, fever, and fatigue.

Chronic sinusitis (e.g., sinusitis lasting more than three months or so) is a highly prevalent disease of particular clinical concern. Chronic sinusitis is the most common chronic medical condition affecting people between the ages of 18-44 years. In 2006, there were an estimated 30.7 million non-institutionalized U.S. adults diagnosed with chronic sinusitis, representing 14% of the population. Pleis, J. R., et al. Vital Health Stat., 10:1-153 (2006). In recent years, between 18 and 22 million physicians office visits per year, and over 5 billion estimated annual dollars in direct health care expenditures were attributable to chronic sinusitis. Ray, N. F., et al. J. Allergy Clin. Immunol., 103:408-414 (1999). The indirect costs of chronic sinusitis also appear to be significant, with the total number of restricted activity days due to chronic sinusitis rising from approximately 50 million per year in the 1980's to approximately 73 million per year in the 1990's.

Sinusitis can have a variety of etiologies (also referred to as causative agents), including bacterial infection, viral infection, fungi (molds), allergies, nasal polyps, or combinations thereof. In spite of the varied potential causes of sinusitis, initial clinical treatments are often not specific for the root cause of the sinusitis. Initial treatments for sinusitis typically involve drug therapy, such as treatment with an anti-inflammatory agent (e.g., a steroid) to reduce inflammation and/or broad spectrum antibiotic therapy to treat infection. However, this initial course of treatment is typically only effective in the case of sinusitis involving a bacterial infection. As a result, many patients suffering from sinusitis are unnecessarily treated with antibiotics and steroids.

The unnecessary administration of steroids and antibiotics in these patients has significant consequences. The administration of antibiotics in the absence of a bacterial infection can lead to the emergence of antibiotic-resistant bacterial strains. In addition, steroid use has been linked to aseptic necrosis of the hip, visual impairment from unknown or unreported glaucoma, and other severe morbidities, in addition to the known and common side effects of oral steroid use.

More effective methods for diagnosing the causative agent of sinusitis offer the promise for improvements in therapy. By diagnosing the root cause of sinusitis, treatment can be selected to specifically address the underlying cause of the sinusitis. Rapid and accurate diagnosis of sinusitis can thus improve therapeutic outcomes while eliminating the undesirable consequences of administering ineffective drug therapies.

### SUMMARY

Probes for assaying the presence of a biomarker in the sinus of a patient are provided. The probes comprise an elongate member having a proximal end and a distal end, and a diagnostic element affixed to the distal end of the elongate member. The diagnostic element comprises an immunoassay, such as a lateral flow immunochromatographic assay, for the presence of a biomarker that is characteristic of sinusitis. Generally, presence of the biomarker is associated with one or more causative agents of sinusitis, such as a bacterial infection, viral infection, fungal infection, allergic reaction, or combinations thereof.

The probes permit positioning of the diagnostic element (and thus the immunoassay) near or within the sinus of a patient, such that the immunoassay contacts sinus fluid in the sinus cavity, the sinus ostium, or a region adjacent to the sinus ostium. In some embodiments, the probe can be positioned using a catheter or endoscope. Once positioned, the immunoassay can detect a biomarker characteristic of a causative agent of sinusitis. Generally, the immunoassay indicates the presence of a biomarker via a simple response that can be observed without the use of instrumentation, such as a change in color. The probes can therefore be used to determine the underlying cause of sinusitis in a patient.

Also provided are in vivo methods of detecting a biomarker in the sinus of a patient. These methods involve contacting sinus fluid in the sinus of a patient with an immunoassay selective for a biomarker associated with sinusitis. Generally, the immunoassay is introduced into the sinus of the patient via the probes described herein. These methods provide a means of rapidly and accurately determining the causative agent of sinusitis.

Also provided are methods of treating sinusitis by administering to a patient a therapeutic regimen selected in view of a biomarker detected in the sinus of the patient. As the biomarker is associated with one or more causative agents of sinusitis, a therapy effective to treat the underlying causative agent can be selected.

### BRIEF DESCRIPTION OF THE FIGURES

FIG. 1 is a schematic drawing illustrating the anatomical tissue structures associated with sinusitis.

FIG. 2A is a perspective view of a probe for diagnosing sinusitis.

FIG. 2B is a schematic view illustrating the deployment of the probe into the maxillary sinus cavity of a patient.

FIG. 3A is a cut-away, schematic top view of an example lateral flow immunochromatographic assay.

FIG. 3B is cross-sectional view of an example lateral flow immunochromatographic assay.

FIG. 4A illustrates a probe which includes a protective sheath. The probe is illustrated with the diagnostic element in the fully retracted position within the protective sheath.

FIG. 4B illustrates a probe which includes a protective sheath. The probe is illustrated with the diagnostic element in the extended position.

FIG. 4C is a schematic view illustrating the deployment of the probe shown in FIGS. 4A and 4B into the maxillary sinus cavity of a patient.

FIG. 5 illustrates a system comprising a probe in combination with a suitable catheter for introducing the probe into a sinus of a patient.

FIG. 6 is a schematic view illustrating the deployment of the system of FIG. 5 into the maxillary sinus cavity of a patient.

FIG. 7 illustrates a system comprising a probe in combination with a suitable dilating catheter for introducing the probe into a sinus of a patient.

FIG. 8 is a schematic view illustrating the deployment of the system of FIG. 7 into the maxillary sinus cavity of a patient.

FIG. 9 illustrates a system comprising a probe in combination with a suitable endoscope for introducing the probe into a sinus of a patient.

FIG. 10 is a schematic view illustrating the deployment of the system of FIG. 9 into the maxillary sinus cavity of a patient.

### DETAILED DESCRIPTION

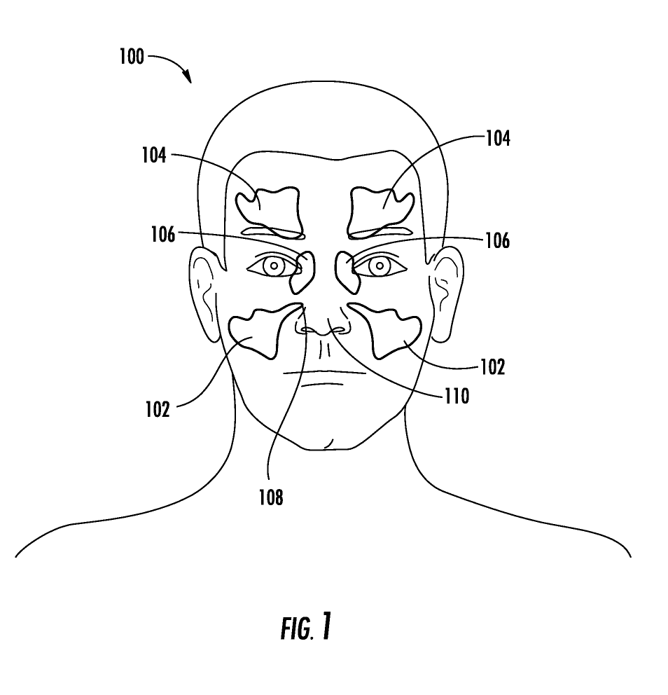

To facilitate understanding of the physiology associated with the devices, systems, kits, and methods described herein, FIG. 1 illustrates the anatomical tissue structures (100) associated with sinusitis. There are four different pairs of sinuses within the nose and face: the maxillary sinuses (102), the frontal sinuses (104), the ethmoid sinuses (106), and the sphenoidal sinuses (not illustrated in FIG. 1; located further towards the back of the head than the other sinuses).

Normally the sinuses are filled with air; however, when the ostium or regions near the ostium of a sinus (such as the ostium of a maxillary sinus, 108) become inflamed or blocked, the flow of mucus from the sinus into nasal passage (110) is interrupted. This can lead to an inflammation or infection of the sinus, termed sinusitis. Sinusitis can be acute or chronic. Acute sinusitis usually lasts for approximately three weeks, but can persist for as long as three months. Chronic sinusitis can last for periods of longer than three months.

Provided is a probe for diagnosing sinusitis. In some embodiments, diagnosing sinusitis involves performing an immunoassay to detect the presence of a biomarker that is associated with a causative agent of sinusitis. Thus, diagnosis can involve determining the causative agent of sinusitis. In some embodiments, diagnosis is performed to determine the causative agent of sinusitis prior to treatment. Treatment regimes can then be selected in view of the causative agent identified. A diagnosis can also performed after an initial diagnosis, during treatment, and/or following treatment, for example, to monitor the progression of sinusitis or treatment efficacy.

The probe is designed to be deployed through the nares of a patient, and into a sinus cavity, a sinus ostium, or to a region adjacent to a sinus ostium. The probe is configured to deliver a diagnostic element to one or more of these regions to determine the underlying cause or causes of sinusitis.

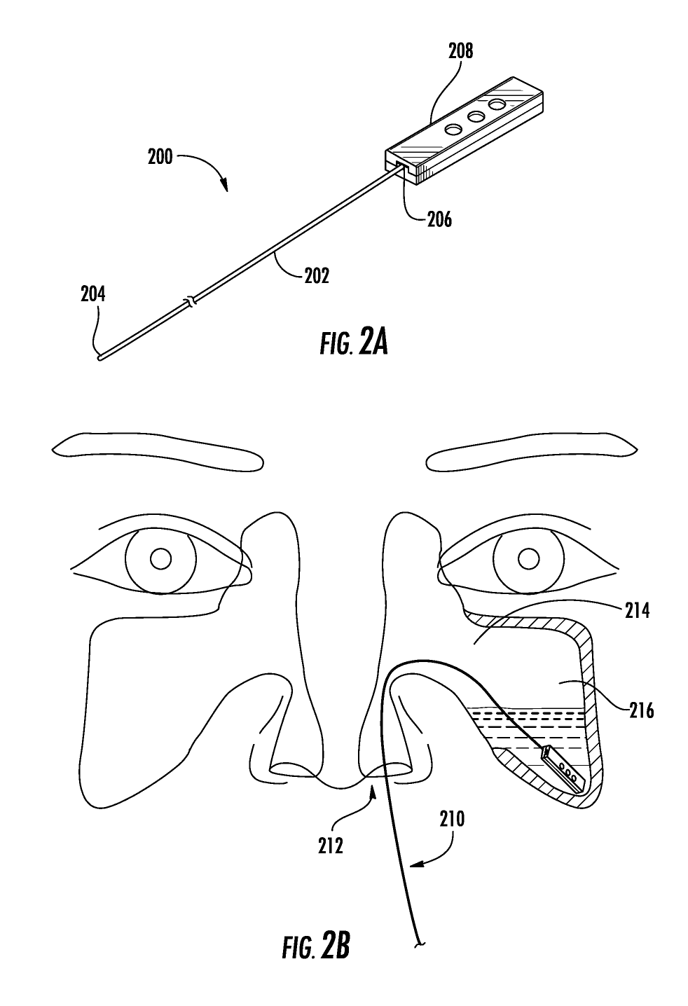

An example probe is illustrated in FIG. 2A. The probe (200) comprises an elongate member (202) having a proximal end (204) and a distal end (206), and a diagnostic element (208) affixed to the distal end of the elongate member. The diagnostic element comprises one or more assays for diagnosing an underlying cause of sinusitis. In some embodiments, the diagnostic element comprises an immunoassay for the presence of a biomarker that is characteristic of sinusitis. In certain embodiments, the diagnostic element comprises an immunoassay for a biomarker that is characteristic of bacterial sinusitis, such as OMP P2, OMP P5, or a combination thereof.

Elongate Member

The elongate member is structured to accommodate the natural sinus geometry of a patient, including the maxillary sinus and/or maxillary sinus ostium, in terms of its dimensions (e.g., length and cross-sectional dimensions), configurations, and operability.

As shown in FIG. 2B, in some embodiments, the probe (210) is configured to be inserted through the nares of a patient (212), and through the ostium of the maxillary sinus of a patient (214), and into the maxillary sinus of the patient (216).

In some embodiments, the elongate member is a wire-like structure having a cross-sectional dimension, length, and flexibility which is suitable to assist in insertion of the probe through the nares and into the maxillary sinus of a patient and/or the probe's subsequent removal from the maxillary sinus of a patient.

In some embodiments, the cross-sectional dimension (e.g., the diameter or thickness) of the elongate member is configured to accommodate the natural interior dimensions of the maxillary sinus ostium to permit entry and movement of the elongate member within the sinus structure. In some cases, the largest cross-sectional dimension of the elongate member is about 5.0 mm or less (e.g., about 4.5 mm or less, about 4 mm or less, about 3.5 mm or less, about 3 mm or less, about 2.5 mm or less, about 2 mm or less, about 1.5 mm or less, or about 1.0 mm or less). The largest cross-sectional dimension of the elongate member can be at least about 0.5 mm (e.g., at least about 1.0 mm, at least about 1.5 mm, at least about 2.0 mm, at least about 2.5 mm, or at least about 3.0 mm) These dimensions are provided with the proviso that the cross-sectional dimensions and composition of the elongate member are selected such that the structural integrity of the elongate member required for probe function is not substantially compromised by the cross-sectional dimension of the elongate member.

The largest cross-sectional dimension of the elongate member can optionally range from any of the minimum dimensions described above to any of the maximum dimensions described above. In some embodiments, the largest cross-sectional dimension of the elongate member is from about 0.5 mm to about 5 mm.

The elongate member is generally of sufficient length to reach a sinus cavity, a sinus ostium, or a region adjacent to a sinus ostium when inserted through the nares of a patient. The elongate member is also typically long enough such that when the diagnostic element affixed to the distal end of the elongate member is positioned in a sinus cavity, a sinus ostium, or a region adjacent to a sinus ostium, the elongate member extends through the nares to a point outside of the patient's body. In this way, the elongate member remains externally accessible to provide a means of manipulating the probe and/or actuating the elongate member.

Optionally, the elongate member has a length of at least about 5 cm (e.g., at least about 6 cm, at least about 7 cm, at least about 8 cm, at least about 9 cm, at least about 10 cm, at least about 11 cm, at least about 12 cm, at least about 13 cm, at least 14 cm, or at least about 15 cm, or longer). In some embodiments, the elongate member has a length of less than about 30 cm (e.g., less than about 25 cm, less than about 20 cm, less than about 19 cm, less than about 18 cm, less than about 17 cm, less than about 16 cm, less than about 15 cm, less than about 14 cm, less than about 13 cm, less than about 12 cm, or less than about 11 cm). The length of the elongate member can optionally range from any of the minimum dimensions described above to any of the maximum dimensions described above.

In some embodiments, the elongate member is from about 5 cm to about 20 cm in length. In certain embodiments, the elongate member is from about 8 cm to about 15 cm in length. In one embodiment, the elongate member is approximately 10 cm in length.

Typically, at least a portion of the elongate member is flexible. In some instances, the elongate member is flexible along its entire length. In other embodiments, the elongate member is comprises two or more regions having different flexibility.

In certain embodiments, the elongate member comprises a flexible region located at or near the distal end of the elongate member, and a region having greater rigidity than the flexible region (referred to as a rigid region) located at or near the proximal end of the elongate member. In some cases, the flexible region of the elongate member comprises a distal portion of the elongate member, extending proximally from the distal end of the elongate member and having a length of at least about 2 cm (e.g., at least about 6 cm, at least about 7 cm, at least about 8 cm, at least about 9 cm, at least about 10 cm, at least about 11 cm, at least about 12 cm, or longer). The flexible region can have a length of less than

about 15 cm (e.g., less than about 10 cm, less than about 8 cm, less than about 6 cm, less than about 5 cm, less than about 4 cm, or less than about 3 cm).

The elongate member, or a flexible region thereof, can have a flexural stiffness of less than about 500 pounds-force per inch over an elongate member length of one inch (e.g., less than about 400, less than about 300, less than about 250, less than about 200, or less than about 100 pounds-force per inch over an elongate member length of one inch). In certain embodiments, the elongate member, or a region thereof, can be bent without fracture to angle of greater than 30° (e.g., to an angle of greater than 45°, greater than 60°, greater than 70°, greater than 90°, greater than 120°, greater than 135°, greater than 150°, or greater than 180°).

The elongate member, or regions thereof, can be formed from a variety of materials. The material can optionally be selected such that the elongate member has structural integrity sufficient to permit positioning of the diagnostic element within a sinus and permit maneuvering and operation of the probe, while also permitting yielding and bending in response to encountered anatomical barriers and obstacles within the nasal and sinus passageways.

The elongate member can be formed from a material or combination of materials, such as polymers, metals, and polymer-metal composites. In some examples, soft durometer materials are used to form all or part of the elongate member to reduce subject recipient discomfort. Examples of suitable metals include stainless steel (e.g., 304 stainless steel), nickel and nickel alloys (e.g., nitinol or MP-35N), titanium, titanium alloys, and cobalt alloys. Examples of suitable plastics and polymeric materials include, but are not limited to, silastic materials and siliconbased polymers, polyether block amides (e.g., PEBAX®, commercially available from Arkema, Colombes, France), polyimides, polyurethanes, polyamides (e.g., Nylon 6,6), polyvinylchlorides, polyesters (e.g., HYTREL®, commercially available from DuPont, Wilmington, Del.), polyethylenes (PE), polyether ether ketone (PEEK), fluoropolymers such as polytetrafluoroethylene (PTFE), perfluoroalkoxy, fluorinated ethylene propylene, or blends and copolymers thereof. In certain embodiments, the elongate member comprises of two different materials. For example, the elongate member may be formed of a flexible material forming a flexible region of the elongate member and a semi-rigid or rigid material forming a rigid region of the elongate member. In other cases, the elongate member, or region thereof, is formed from a combination of a semi-rigid internal material and a soft, pliable exterior material. Radiopaque alloys, such as platinum and titanium alloys, may also be used to fabricate, in whole or in part, the elongate member to facilitate real-time imaging of probe positioning.

The elongate member can be coated or treated with various polymers or other compounds in order to provide desired handling or performance characteristics, such as to increase lubricity. In certain embodiments, the elongate member is coated with polytetrafluoroethylene (PTFE) or a hydrophilic polymer coating, such as poly(caprolactone), to enhance lubricity and impart desirable handling characteristics.

In some cases, the elongate member is straight (i.e., unbent) when no force is applied to the elongate member. In other cases, one or more preformed bends or curves can be incorporated into the elongate member to facilitate deployment of the device in vivo.

Optionally, an actuating element may be affixed to the proximal end of the elongate member. The actuating element can be a surface or feature, such as a handle, ring, nob, or flange, which is configured to facilitate a user's actuation of the elongate member in the distal and/or proximal directions, configured to facilitate a user's position of the probe within a patient, or combinations thereof.

Diagnostic Element

Probes contain a diagnostic element. Typically, the diagnostic element is affixed to the distal end of the elongate member. For example, the diagnostic element can extend from the distal tip of the elongate member. In other embodiments, the diagnostic element may be integrated within the distal region of the elongate member. For example, the diagnostic element may be contained on or within a surface of the distal end of the elongate member.

Generally, the diagnostic element has a length sufficient to incorporate an immunoassay. For example, the diagnostic element can have a length of at least about 1.0 cm (e.g., at least about 1.5 cm, at least about 2.0 cm, at least about 2.5 cm, at least about 3.0 cm, at least about 3.5 cm, or at least about 4.0 cm). In some embodiments, the diagnostic has a length of less than about 5.0 cm (e.g., less than about 4.5 cm, less than about 4.0 cm, less than about 3.0 cm, less than about 2.5 cm, or less than about 2.0 cm). The length of the diagnostic element can range from any of the minimum dimensions described above to any of the maximum dimensions described above. In some embodiments, the diagnostic element is from about 1.0 cm to about 4.0 cm in length.

The diagnostic element may have a variety of shapes. The shape of the diagnostic element can optionally be determined by a variety of factors including anatomical considerations, and the number and type of immunoassays to be incorporated on the diagnostic element. For example, the diagnostic element can optionally be cuboid, cubic, conical, pyramidal, polygonal, or cylindrical in shape.

In some embodiments, the cross-sectional dimension (e.g., the diameter or thickness) of the diagnostic element is selected in view of the natural interior dimensions of the maxillary sinus ostium to permit entry and movement of the elongate member within the sinus structure. In some cases, the largest cross-sectional dimension of the diagnostic element is about 5.0 mm or less (e.g., about 4.5 mm or less, about 4 mm or less, about 3.5 mm or less, about 3 mm or less, about 2.5 mm or less, about 2 mm or less, about 1.5 mm or less, or about 1.0 mm or less). The largest cross-sectional dimension of the diagnostic element can be at least about 0.5 mm (e.g., at least about 1.0 mm, at least about 1.5 mm, at least about 2.0 mm, at least about 2.5 mm, or at least about 3.0 mm) The largest cross-sectional dimension of the diagnostic element can range from any of the minimum dimensions described above to any of the maximum dimensions described above. In some embodiments, the largest cross-sectional dimension of the diagnostic element is from about 0.5 mm to about 5 mm.

The diagnostic element contains an immunoassay for the presence of a biomarker that is characteristic of sinusitis. Biomarkers are naturally occurring compounds that are characteristic of a particular pathological or physiological process. Accordingly, biomarkers may be assayed to identify risk for, diagnosis of, or progression of a pathological or physiological process. Suitable biomarkers include proteins, hormones, prohormones, lipids, carbohydrates, DNA, RNA, and combinations thereof.

The immunoassay can detect and/or quantify one or more biomarkers that are associated with a particular form of sinusitis. For example, the immunoassay can detect and/or quantify biomarkers that are associated with a bacterial infection, viral infection, fungal infection, allergic response, or combinations thereof. By detecting and/or quantifying one or more of these biomarkers in sinus, the underlying etiology of sinusitis in a patient can be accurately determined.

In some embodiments, the diagnostic element contains an immunoassay for the presence of a biomarker that is characteristic of bacterial sinusitis. Due to unique growth characteristics, bacterial biofilms produce a distinct set of proteins that may be used to distinguish between commensal and pathogenic states. The immunoassay can be used to detect and/or quantify signature proteins that distinguish specific bacterial pathogens from typically sterile sites in the paranasal sinus cavities. The immunoassay can detect and/or quantify one or more of these proteins. In some embodiments, the immunoassay may be designed to detect and/or quantify one or more of these proteins, so as to identify the sinusitis as bacterial sinusitis. In certain embodiments, the immunoassay may be designed to detect and/or quantify one or more of these proteins, so as to identify the specific bacterial pathogens (e.g., the bacterial strain) associated with the bacterial sinusitis.

For example, biofilms produced by nontypeable Haemophilius influenzae (NTHI) generate a specific protein profile; specifically, outer membrane proteins (OMPs) are predominant components of the NTHI biofilm supernatant. Of particular interest are major OMPs associated with bacterial virulence: outer membrane protein P5 (OMP P5, Accession No. YP_{—}248824) and outer membrane protein P2 (OMP P2, Accession No. YP_{—}247859). In some embodiments, the diagnostic element contains an immunoassay for the presence of OMP P2, OMP P5, or combinations thereof.

Suitable immunoassays for the detection and or quantification of biomarkers optionally indicate the presence and/or quantity of a biomarker by a colorimetric and/or fluorometric response. In certain embodiments, the outcome of the immunoassay (e.g., the presence and/or quantity of a biomarker) can be determined without the aid of instrumentation. For example, the presence and/or quantity of a biomarker can be determined by observing a color change.

The diagnostic element can be a lateral flow immunochromatographic assay (also known as a lateral flow immunoassay). Lateral flow immunochromatographic assays are known in the art, and are used, for example, as pregnancy tests and to test for streptococcal colonization. See also, for example, U.S. Pat. No. 6,485,982 to Charlton.

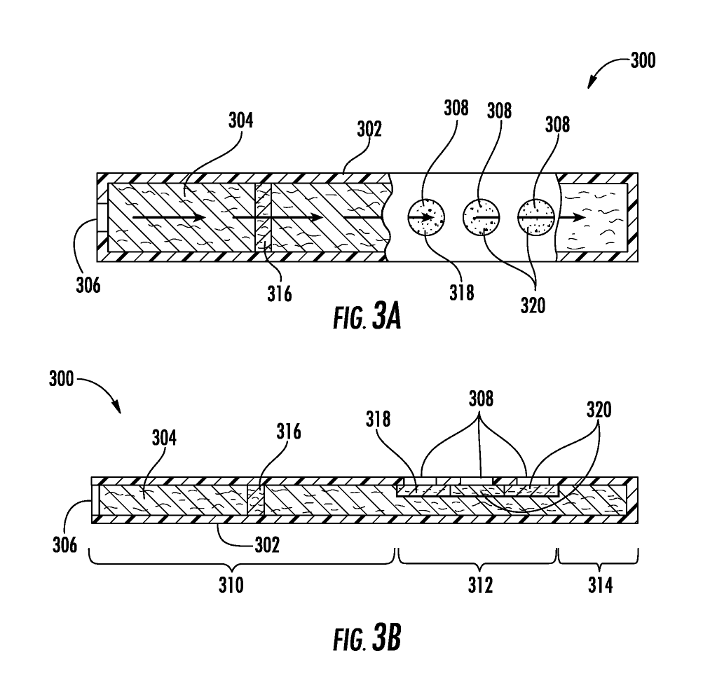

Referring to the drawings, FIGS. 3A and 3B are schematic drawings that illustrate a lateral flow immunoassay (300) that can be used in the disclosed probes. In other embodiments, the immunoassays described below can be used independent from the probes, for example, for the in vitro characterization of sinus fluid.

The lateral flow assay comprises an outer casing 302 which defines a hollow, elongate enclosure filled with a permeable, sorbent material 304 (such as a cellulose membrane strip). The casing 302 also defines a test liquid inlet 306. The casing 302 may be transparent or opaque. In cases where the casing 302 is opaque, the casing optionally contains openings or windows 308 through which the sorbent material 304 is visible. The casing 302 may be made of any inert, non-absorbent material. Suitable casing materials include polymers, silicones, glasses, metals, ceramics, inorganic materials, and combinations thereof.

In some embodiments, the casing is formed at least in part from an inert, non-absorbent polymer such as a polyether block amide (e.g., PEBAX®, commercially available from Arkema, Colombes, France), a polyimide, polyurethane, polyamide (e.g., Nylon 6,6), polyvinylchloride, polyester, (HYTREL®, commercially available from DuPont, Wilmington, Del.), polyethylene (PE), polyether ether ketone (PEEK), fluoropolymers such as polytetrafluoroethylene (PTFE), perfluoroalkoxy, fluorinated ethylene propylene, or a blend or copolymer thereof. Silastic materials and siliconbased polymers can also be used. In some embodiments, the casing is formed at least in part from a metal, such as stainless steel.

The sorbent material 304 may be, for example, a hydrophobic nitrocellulose or cellulose acetate membrane. The sorbent material 304 and the interior of casing 302 together define a flow path passing generally from left to right in FIGS. 3A and 3B. When the lateral flow immunoassay is placed with inlet 306 disposed within or otherwise in contact with a liquid sample (such as sinus fluid), the liquid is transported by capillary action, wicking, or simple wetting along the flow path through downstream flow section 310, test section 312, and into reservoir section 314, generally as depicted by the arrows.

Disposed within the flow section 310 of the sorbent material is a conjugate band 316 of dehydrated conjugate, e.g., an antibody conjugated to metal or colored particles (a marker substance). The particles that can be used are not limited to any particular materials, and can comprise metals such as colloidal gold, natural polymers such as latex (e.g., latex particle, latex microparticle), polymer microspheres or microbeads, quantum dots, magnetic particles, and glass beads. As the liquid sample moves past the conjugate band 316, the conjugate is entrained in the liquid, reconstituted, and reacts or competes with analyte (e.g., proteins within the protein profile of a pathogenic bacteria of interest) within the liquid sample, if present.

A control band 318 and one or more test bands 320 are disposed within the test section 312 of the sorbent material 304. In the drawing, the control band 318 and test band 320 are illustrated as being disposed serially along the flow path. Alternatively, the control band 318 and test band 320 may be disposed side by side or in other spatial relationships. The one or more test bands 320 comprise a preselected quantity of capture agent (e.g., an antibody) that specifically binds an epitope of the analyte to be detected immobilized in place within the flow path. The control band 318 can comprise a capture agent (e.g., an antibody) that binds a control analyte to indicate to the user that the test was successfully run.

Disposed beyond the test section 312 is a reservoir section (314) comprising a relatively large mass of sorbent or supersorbent material. The purpose of reservoir section 314 is to assure that a reasonably large amount of test liquid is drawn through test section 312. Increasing the volume of reservoir section 314 can have the effect of increasing the sensitivity of the assay procedure, as it results in an increase in the amount of ligand passing through the test section 312. Suitable sorbents include commercial materials of the type available, for example, from The Dow Chemical Company of Midland, Mich., and the Chemical division of American Colloid, Arlington Heights, Ill. These materials can absorb many times their weight in water and are commonly used in disposable diapers. They comprise lightly crosslinked polyacrylate salts, typically alkali metal salts.

Metal sols and other types of colored particles also useful as marker substances in immunoassay procedures are also known in the art. See, for example, U.S. Pat. No. 4,313,734, Feb. 2, 1982, to Leuvering, the disclosure of which is incorporated herein by reference. For details and engineering principles involved in the synthesis of colored particle conjugates see Horisberger, “Evaluation of Colloidal Gold as a Cytochromic Marker for Transmission and Scanning Electron Microscopy,” Biol. Cellulaire, 36:253-258 (1979); Leuvering, et al., “Sol Particle Immunoassay,” J. Immunoassay 1:77-91 (1980), and Frens, “Controlled Nucleation for the Regulation of the Particle Size in Monodisperse Gold Suspensions,” Nature, Physical Science, 241:20-22 (1973). Fluorescent and magnetic labeled particles may also be used. In these instances, additional devices, such as a blacklight, may be used to determine the location of particles within the assay.

The binding agent and capture agent selectively bind on or more biomarkers of interest, such as one or more proteins within the protein profile of a pathogenic bacteria of interest. For example, the device may contain a binding agent and capture agent pair that selectively bind outer membrane protein P5 (OMP P5), a binding agent and capture agent pair that selectively bind outer membrane protein P2 (OMP P2), or a combination thereof.

Suitable capturing and/or detecting agents include antibodies, ligands, such as natural receptors, aptamers, peptides, and small molecule ligands that specifically bind the target analyte. The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen. The term encompasses intact and/or full length immunoglobulins of types IgA, IgG (e.g., IgG1, IgG2, IgG3, IgG4), IgE, IgD, IgM, IgY, antigen-binding fragments or single chains of complete immunoglobulins (e.g., single chain antibodies, Fab fragments, F(ab′)2 fragments, Fd fragments, scFv (single-chain variable), and dAb fragments), and other proteins that include at least one antigen-binding immunoglobulin variable region, e.g., a protein that comprises an immunoglobulin variable region, e.g., a heavy (H) chain variable region (VH) and a light (L) chain variable region (VL). The light chains of an antibody may be of type kappa or lambda. An antibody may be polyclonal or monoclonal. A polyclonal antibody contains immunoglobulin molecules that differ in sequence of their complementarity determining regions (CDRs) and, therefore, typically recognize different epitopes of an antigen. Often a polyclonal antibody is derived from multiple different B cell lines each producing an antibody with a different specificity. A polyclonal antibody may be composed largely of several subpopulations of antibodies, each of which is derived from an individual B cell line. A monoclonal antibody is composed of individual immunoglobulin molecules that comprise CDRs with the same sequence, and, therefore, recognize the same epitope (i.e., the antibody is monospecific). Often a monoclonal antibody is derived from a single B cell line or hybridoma. An antibody may be a “humanized” antibody in which for example, a variable domain of rodent origin is fused to a constant domain of human origin or in which some or all of the complementarity-determining region amino acids often along with one or more framework amino acids are “grafted” from a rodent, e.g., murine, antibody to a human antibody, thus retaining the specificity of the rodent antibody.

In certain embodiments, the diagnostic element comprises a lateral flow immunochromatographic assay affixed to the distal end of the elongate member, such that the test liquid inlet for the lateral flow immunochromatographic assay is positioned at the distal end or tip of the diagnostic element.

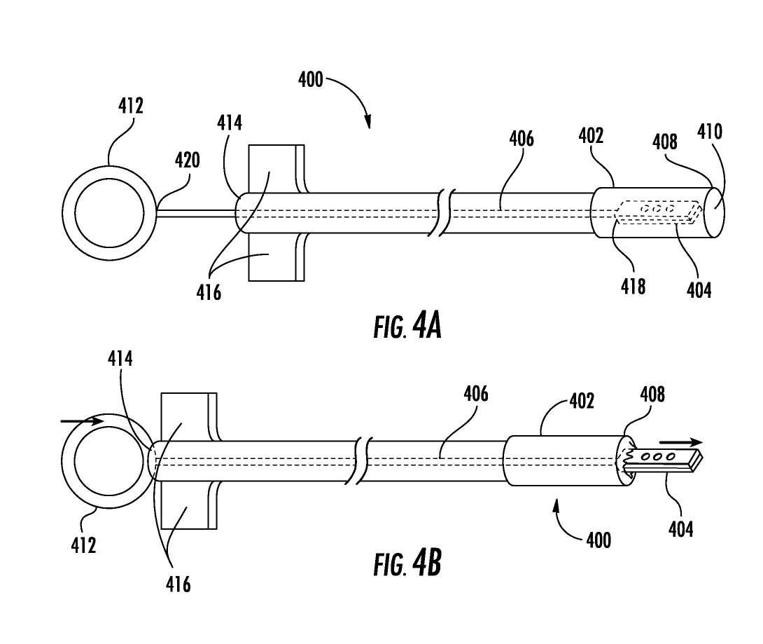

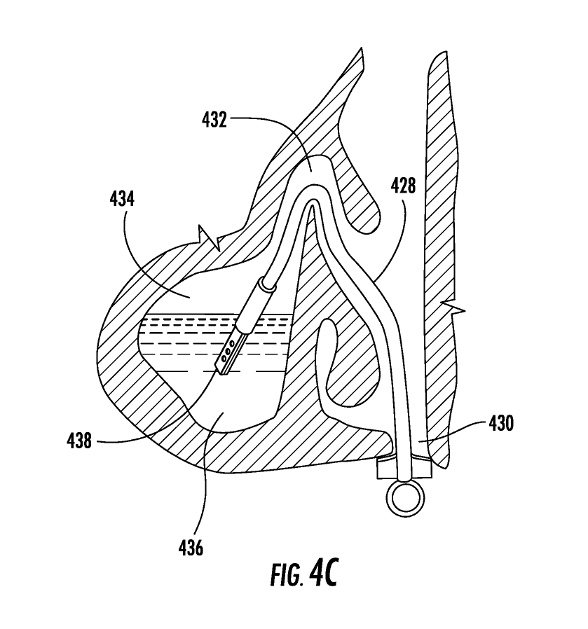

Referring now to FIG. 4A, in some embodiments, the probe (400) can further comprising a hollow protective sheath (402) that surrounds the diagnostic element (404), such that the diagnostic element is retractably positioned within the protective sheath. As shown in FIG. 4B, actuation of the elongate member (406) in the distal direction (indicated by the arrows) extends at least a portion of the diagnostic element beyond the distal end of the protective sheath (408). The elements of the probe can be configured such that the, upon actuation of the elongate member in the distal direction, the distal tip of the diagnostic element is extended beyond the distal end of the protective sheath by at least 0.25 cm (e.g., by about 0.5 cm, by about 1.0 cm, by about 1.5 cm, by about 2.5 cm, or by about 3.0 cm beyond the distal end of the protective sheath).

The protective sheath can be formed from a material or combination of materials, such as polymers, metals, and polymer-metal composites. In some examples, soft durometer materials are used to form all or part of the protective sheath to reduce subject recipient discomfort. Examples of suitable metals include stainless steel (e.g., 304 stainless steel), nickel and nickel alloys (e.g., nitinol or MP-35N), titanium, titanium alloys, and cobalt alloys. Examples of suitable plastics and polymeric materials include, but are not limited to, silastic materials and siliconbased polymers, polyether block amides (e.g., PEBAX®, commercially available from Arkema, Colombes, France), polyimides, polyurethanes, polyamides (e.g., Nylon 6,6), polyvinylchlorides, polyesters (e.g., HYTREL®, commercially available from DuPont, Wilmington, Del.), polyethylenes (PE), polyether ether ketone (PEEK), fluoropolymers such as polytetrafluoroethylene (PTFE), perfluoroalkoxy, fluorinated ethylene propylene, or blends and copolymers thereof. Radiopaque alloys, such as platinum and titanium alloys, may also be used to fabricate, in whole or in part, the protective sheath to facilitate real-time imaging of probe positioning.

One or more surfaces of the protective sheath can be coated or treated with various polymers or other compounds in order to provide desired handling or performance characteristics, such as to increase lubricity. For example, the interior surface of the protective sheath can be coated or treated to facilitate extension and/or retraction of the diagnostic element, actuation of the elongate member within or through the protective sheath, or combinations thereof. Similarly, the exterior surface of the protective sheath can be coated or treated to facilitate advancement of the probe through the nasal passageways to reach a sinus, to facilitate advancement of the probe through the lumen of an accompanying medical device used to deploy the probe, such as a catheter or endoscope, or combinations thereof. Suitable coatings include polytetrafluoroethylene (PTFE) or a hydrophilic polymer coatings, such as poly(caprolactone).

As shown in FIG. 4A, the distal end of the protective sheath (408) can optionally be sealed with a membrane (410) that encloses the distal end of the protective sheath. The membrane can serve to protect and enclose the diagnostic element prior to use, and/or to protect and enclose the diagnostic element during insertion of the probe into the patient (for example, to prevent the immunoassay from contacting a fluid prior to contacting the sinus fluid of interest).

In some cases, the membrane may be removed prior to inserting the probe into the nares of a patient. In other instances, the membrane is retained, and the device is inserted into the patient in its sealed (and fully retracted) configuration. As shown in FIG. 4B, actuation of the elongate member in the distal direction can then extend at least a portion of the diagnostic element beyond the distal end of the protective sheath, puncturing through the membrane. In these embodiments, the elements of the probe, including the membrane, diagnostic element, and elongate member, are selected such that the diagnostic element can readily penetrate through the membrane, where it can subsequently make contact with the sinus fluid.

Suitable membranes include polymer thin films. For example, the membrane can comprise a thin film of an inert, non-absorbent polymer such as a polyether block amide (e.g., PEBAX®, commercially available from Arkema, Colombes, France), a polyimide, polyurethane, polyamide (e.g., Nylon 6,6), polyvinylchloride, polyester, (HYTREL®, commercially available from DuPont, Wilmington, Del.), polyethylene (PE), polyether ether ketone (PEEK), fluoropolymers such as polytetrafluoroethylene (PTFE), perfluoroalkoxy, fluorinated ethylene propylene, or a blend or copolymer thereof.

As shown in FIG. 4A, the protective sheath can proximally extend beyond the proximal end of the diagnostic element (418) to enclose a portion of the elongate member.

In some embodiments, the protective sheath encloses a substantial portion of the elongate member when the diagnostic element is in a fully retracted position within the protective sheath. For example, the protective sheath can enclose at least 50% of the length of the elongate member when the diagnostic element is in a fully retracted position within the protective sheath (e.g., at least 60% of the length, at least 70% of the length, at least 75% of the length, or at least 80% of the length). In certain embodiments, the protective sheath encloses a substantial portion of the elongate member when the diagnostic element is in a fully retracted position within the protective sheath, such that when the probe is inserted into the sinus of a patient, the proximal end of the protective sheath (414) remains outside of the patients body (e.g., it remains positioned outside of the patient's nares).

In some embodiments, the protective sheath can proximally extend beyond the proximal end of the diagnostic element (418) to enclose a portion of the elongate member, such that the distance between the proximal end of the elongate member (420) and the proximal end of the protective sheath (414) is from about 1 cm to about 5 cm when the diagnostic element is in a fully retracted position within the protective sheath (as shown in FIG. 4A).

In some embodiments, an actuating element (412) may be affixed to the proximal end of the elongate member. The actuating element can be a surface or feature, such as a handle, ring, nob, or flange, which is configured to facilitate a user's actuation of the elongate member in the distal and/or proximal directions, configured to facilitate a user's position of the probe within a patient, or combinations thereof. Similar actuating elements can also be positioned at the proximal end of the protective sheath (416).

In certain embodiments, the actuating element (412) has a cross-sectional dimension that is greater than the largest interior cross-sectional dimension of the protective sheath at the sheath's proximal end, such that the actuating element (412) cannot be actuated in the distal direction beyond the proximal end of the protective sheath (e.g., into the protective sheath). In this way, a maximum degree of actuation of the elongate member in the distal direction is defined by the point at which the actuating element contacts the proximal end of the protective sheath. In some embodiments, ribs, protrusions, or other features can extend from the surface of the elongate member between the proximal end of the protective sheath (414) and the proximal end of the elongate member (414). These ribs, protrusions, or other features can function to increase a cross-sectional dimension of the elongate member at a point along the length of the elongate member to an amount greater than the largest interior cross-sectional dimension of the protective sheath at the sheath's proximal end, such that a maximum degree of actuation of the elongate member in the distal direction is defined by the point at which the ribs, protrusions, or other features contacts the proximal end of the protective sheath.

As shown in FIG. 4C, the probe (428) is configured to be inserted through the nares of a patient (430), and through the ostium of the maxillary sinus of a patient (432), and into the maxillary sinus of the patient (434). Generally, the probe is inserted while the diagnostic tip is in the fully retracted within the protective sheath. The elongate member can then be actuated, extending at least a portion of the diagnostic element (438) beyond the distal end of the protective sheath. The diagnostic element contacts sinus fluid (436) in the sinus of the patient, such that the immunoassay for the biomarker is successfully performed.

In some embodiments, the probes described herein can be introduced into the sinus of a patient using a catheter. Accordingly, systems and devices are described that comprise a probe in combination with a suitable catheter for introducing the probe into a sinus of a patient. Catheters for accessing the sinuses of a patient are known in the art. See, for example, U.S. Pat. No. 7,678,099 to Ressemann, et al, and U.S. Patent Application Nos. US 2009/0187098 to Makower, et al. and US 2010/0174308 to Chang, et al. These catheters can be modified to provide delivery of the probes, for example, by modifying the lumen so that it is sized and configured such that the probes can be advanced through the lumen to expose a distal portion of the probes beyond the distal end of the catheter.

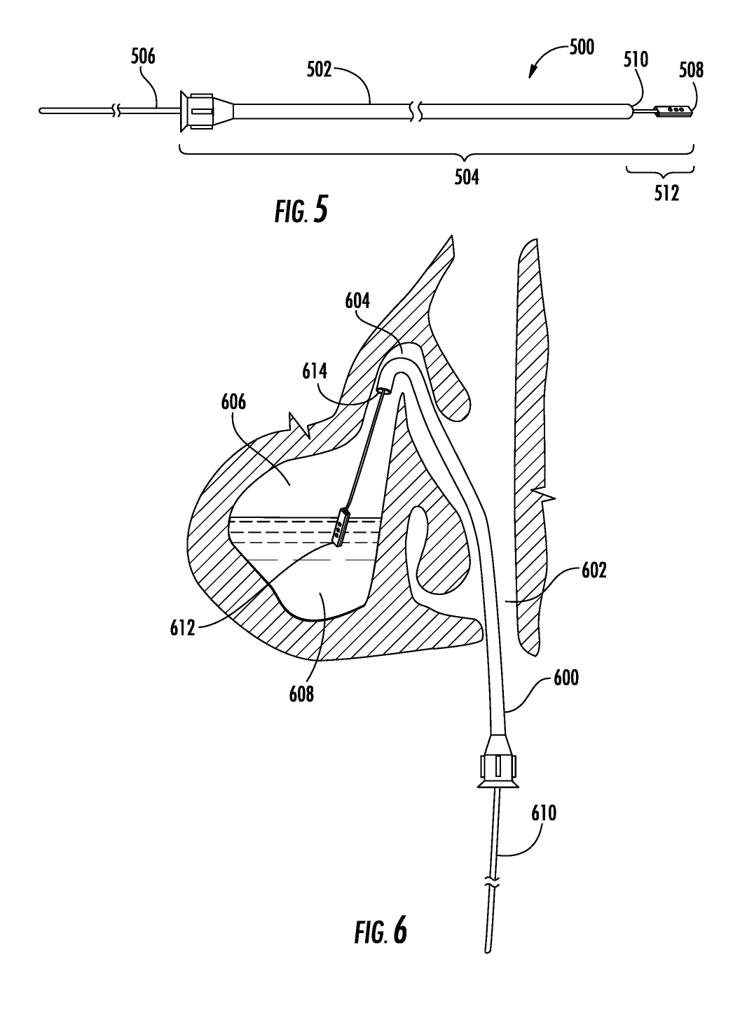

An example system is illustrated in FIG. 5. The system (500) includes a catheter (502) and a probe (506). The catheter includes a lumen that is sized and configured such that the probe can be advanced through the lumen to expose a distal portion of the probe (512) beyond the distal end of the catheter (510). For example, the lumen may have a minimum cross-sectional dimension greater than the maximal cross-sectional dimension of a distal region of the probe (504), such that the probe can be advanced through the catheter to extend at least a portion of the diagnostic element (508) beyond the distal end of the catheter (510). The system can optionally further comprise a guidewire for positioning the catheter.

Referring to FIG. 6, systems and devices comprising a catheter (600) can be used to insert the distal end of a probe (610) into a sinus of a patient. The catheter (600) is configured to be inserted through the nares of a patient (602), and through the ostium of the maxillary sinus of a patient (604), and into the maxillary sinus of the patient (606). The catheter can optionally be positioned using a guidewire. The probe (610) can then be advanced through the lumen of the catheter, such that at least a portion of the diagnostic element (612) beyond the distal end of the catheter (614). In this way, the diagnostic element is brought into contact with sinus fluid (608) in the sinus of the patient, such that the immunoassay for the biomarker is successfully performed.

The catheter can be composed of a semi-rigid, flexible material having structural integrity sufficient to permit positioning within a sinus, and maneuvering and operation of the probe, while permitting yielding and bending in response to encountered anatomical barriers and obstacles within the nasal and sinus passageways. Suitable materials include, but are not limited to, plastics and polymeric materials. Examples of suitable plastics and polymeric materials include, but are not limited to, silastic materials and siliconbased polymers, polyurethane, and the like. In some examples, soft durometer materials are used for the catheter to reduce subject recipient discomfort. In some examples, the catheter can be composed of two different materials, such as the combination of a semi-rigid internal material and a soft, pliable exterior material. The dimensions of the catheter can be selected in view of the probe to be used, as well as anatomical constraints.

The catheter may be rendered steerable or volitionally bendable. Steerable catheters may utilize mechanical steering assemblies (e.g., pull wires, hinges, etc.) or shape memory materials (e.g., nickel titanium alloys, shape memory polymers, etc.) to induce the device to undergo the desired bending or curvature after it has been inserted into the body. Steerable and volitionally bendable catheters and devices are known in the art. See, for example, U.S. Pat. No. 5,507,725 (Savage et al.); U.S. Pat. No. 5,656,030 (Hunjan et al.); U.S. Pat. No. 6,183,464 (Webster); U.S. Pat. No. 5,251,092 (Qin et al.); U.S. Pat. No. 6,500,130 (Kinsella et al.); U.S. Pat. No. 6,571,131 (Nguyen); U.S. Pat. No. 5,415,633 (Lazarus et al.); U.S. Pat. No. 4,998,916 (Hammerslag et al.); U.S. Pat. No. 4,898,577 (Badger et al.); U.S. Pat. No. 4,815,478 (Buchbinder et al.); and published United States Patent Application Nos. 2003/0181827A1 (Hojeibane et al.) and 2003/0130598A1 (Manning et al.).

In some embodiments, catheter is a dilating catheter. Dilating catheters contain a dilating member, such as a balloon, integrated at or within the distal end of the catheter. The dilating catheter is configured such that when the catheter is inserted into a sinus of the patient, the dilating member is positioned within or in proximity to the ostium of the sinus. For example the dilating member may positioned within about 5 cm of the distal end of the dilating catheter. Dilation of the dilating member can then dilate open a narrowed region of an ostium. Dilating catheters for accessing the sinuses of a patient are known in the art. See, for example, U.S. Pat. No. 7,678,099 to Ressemann, et al, and U.S. Patent Application Nos. US 2009/0187098 to Makower, et al. and US 2010/0174308 to Chang, et al. These dilating catheters can be modified to provide delivery of the probes, for example, by modifying the lumen so that it is sized and configured such that the probes can be advanced through the lumen to expose a distal portion of the probes beyond the distal end of the catheter.

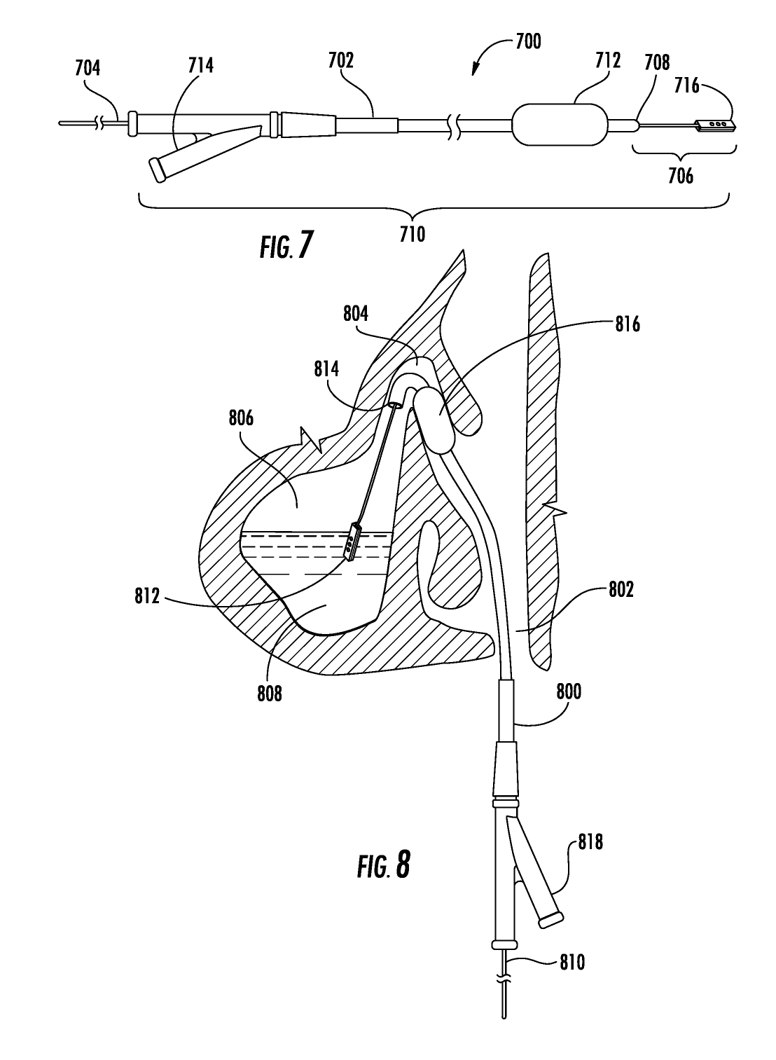

An example system including a probe and a dilating catheter is illustrated in FIG. 7. The system (700) includes a dilating catheter (702) containing a dilating member (712), and a probe (704). The dilating catheter includes a lumen that is sized and configured such that the probe can be advanced through the lumen to expose a distal portion of the probe (706) beyond the distal end of the dilating catheter (708). For example, the lumen may have a minimum cross-sectional dimension greater than the maximal cross-sectional dimension of a distal region of the probe (710), such that the probe can be advanced through the catheter to extend at least a portion of the diagnostic element (716) beyond the distal end of the catheter (708). The dilating catheter further includes an inflation port (714) at the proximal end of the catheter that can be used to inflate and deflate the dilating member (712). The inflation port may be configured to connect to, for example a syringe, by means of a Leur lock or other connection to facilitate inflation and deflation of the dilating member. The system can optionally further comprise a guidewire for positioning the catheter within the sinus of a patient.

Referring to FIG. 8, systems and devices comprising a dilating catheter (800) can be used to insert the distal end of a probe (810) into a sinus of a patient. The catheter (800) is configured to be inserted through the nares of a patient (802), and through the ostium of the maxillary sinus of a patient (804), and into the maxillary sinus of the patient (806). The catheter can optionally be positioned using a guidewire. The probe (810) can then be advanced through the lumen of the catheter, such that at least a portion of the diagnostic element (812) beyond the distal end of the catheter (814). In this way, the diagnostic element is brought into contact with sinus fluid (808) in the sinus of the patient, such that the immunoassay for the biomarker is successfully performed. The dilating member (816) is positioned within or in proximity to the ostium of the sinus. The inflation port (818) at the proximal end of the catheter remains outside the nares of the patient. The inflation port (818) can be used to inflate and deflate the dilating member (816), dilating open a narrowed region of the sinus ostium (804).

In some embodiments, the probes described herein can be introduced into the sinus of a patient using an endoscope. Accordingly, systems and devices are described that comprise a probe in combination with a suitable endoscope for introducing the probe into a sinus of a patient. Endoscopes that can be used within the nasal passageways and/or to image the sinus are known in the art. See, for example, U.S. Pat. No. 7,678,099 to Ressemann, et al. These endoscopes can be modified to provide delivery of the probes, for example, by modifying incorporating a lumen extending from the proximal end of the endoscope to the distal end of the endoscope that it is sized and configured such that the probes can be advanced through the lumen to expose a distal portion of the probes beyond the distal end of the endoscope.

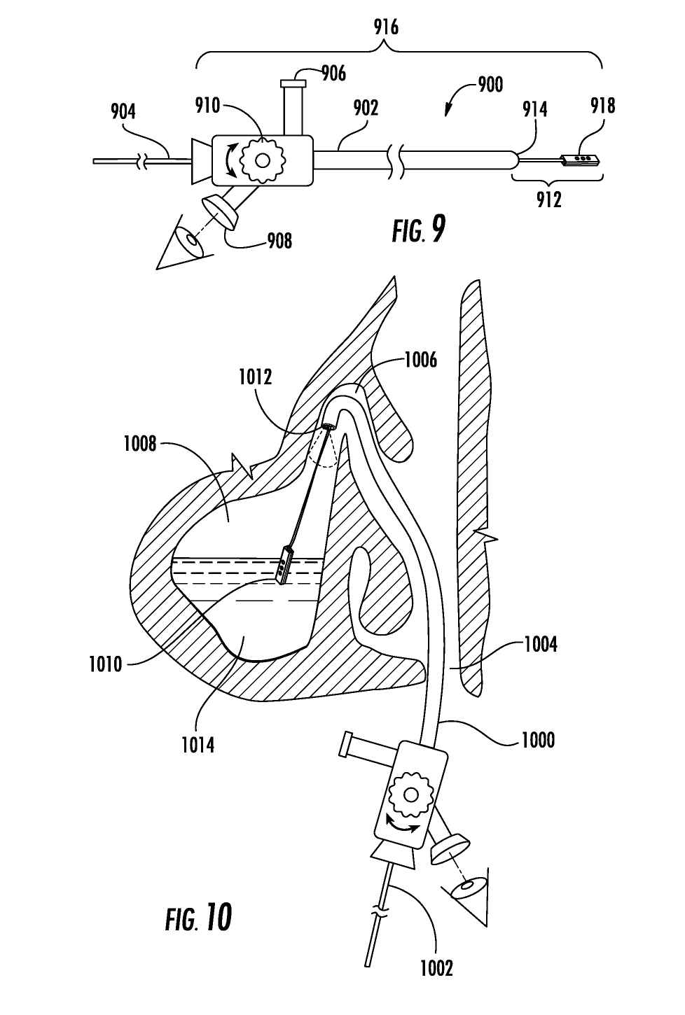

An example system including a probe and an endoscope is illustrated in FIG. 9. The system (900) includes an endoscope (902) and a probe (904). The endoscope (902) can include a fiber optic inlet (906) that can provide illumination at the distal end of the endoscope. The endoscope (902) can also include a visualization scope (908) that can be used to visualize an imaging field extending beyond the distal end of the endoscope. The tip of the endoscope can be configured to be steerable, in which case a deflection nob (910), positioned at the proximal end of the endoscope, can be used to deflect and steer the endoscope tip and/or direct the imaging field extending beyond the distal end of the endoscope.

The endoscope (902) includes a lumen that is sized and configured such that the probe can be advanced through the lumen to expose a distal portion of the probe (912) beyond the distal end of the endoscope (914). For example, the lumen may have a minimum cross-sectional dimension greater than the maximal cross-sectional dimension of a distal region of the probe (916), such that the probe can be advanced through the catheter to extend at least a portion of the diagnostic element (918) beyond the distal end of the catheter (914).

Referring to FIG. 9, systems and devices comprising an endoscope (1000) can be used to insert the distal end of a probe (1002) into a sinus of a patient. The endoscope (1000) is configured to be inserted through the nares of a patient (1004), and optionally through the ostium of the maxillary sinus of a patient (1006), and optionally into or in proximity to the maxillary sinus of the patient (1008). The probe (1002) can then be advanced through the lumen of the endoscope, such that at least a portion of the diagnostic element (1010) beyond the distal end of the endoscope (1012). In this way, the diagnostic element is brought into contact with sinus fluid (1014) in the sinus of the patient, such that the immunoassay for the biomarker is successfully performed. The endoscope may be used to facilitate insertion of the probe, to image the sinus ostium and/or sinus cavity, or combinations thereof.

The probes and systems described herein can be packaged in kits for use in primary care settings in combination with instructions for use. The probes described herein can also be packed in kits for sale in a pharmacy in combination with instructions for use. In some embodiments, the kit can be utilized by a patient to self-diagnose the cause of sinusitis. In these instances, the patient can then convey the results of the assay to a health care provider, who can prescribe appropriate therapeutic measures to address the underlying cause of the sinusitis.

Also provided are methods of diagnosing the cause of sinusitis in a patient. These methods can involve contacting sinus fluid within a sinus of the patient (i.e., in vivo) with an immunoassay for a biomarker associated with one or more causative agents of sinusitis, such as a bacterial infection, viral infection, fungal infection, allergic reaction, or combinations thereof. In certain embodiments, the sinus is a patient's maxillary sinus. In some embodiments, the biomarker is selected from the group consisting of OMP P2, OMP P5, or a combination thereof, and the presence of OMP P2, OMP P5, or a combination thereof is characteristic of bacterial sinusitis.

Methods for in vivo detection of a biomarker in the sinus of a patient can involve advancing an immunoassay for a biomarker into the sinus of the patient, contacting sinus fluid in the sinus of the patient with the immunoassay, retracting the immunoassay, and analyzing the immunoassay to determine if the biomarker is present in the sinus of the patient.

Generally, the sinus fluid is contacted by an immunoassay using the probes described herein. Accordingly, these methods can involve advancing the probes described herein into the sinus of a patient, such that the diagnostic element of the probe contacts sinus fluid in the sinus, sinus ostium, or regions adjacent to the sinus ostium of a patient. In some embodiments, the probes can be introduced into the sinus of a patient using an catheter or endoscope, as described above. Probes can also be deployed into a sinus with the aid of a separate imaging instrument, such as a rhinoscope or endoscope, to image probe advancement and placement.

The probes, systems, and devices can be used to provide a point-of-care means of determining the underlying causes of sinusitis. Preferably, the methods and tests described herein allow a clinician to receive critical information regarding the underlying cause of sinusitis immediately, for example in the clinic or at the patient's bedside.

Also provided are methods of treating sinusitis. These methods can involve administering to the patient a therapeutic regimen selected in view of a biomarker detected in the sinus of the patient. The therapeutic regimen can be selected so as to be effective to treat one or more causative agents of sinusitis, such as a bacterial infection, viral infection, fungal infection, allergic reaction, or combinations thereof. For example, if the immunoassay registers the presence of a biomarker associated with bacterial sinusitis, the method of treatment can involve administration of an antibiotic. Suitable therapeutic compounds that can be used to treat bacterial sinusitis include antibiotics such as penicillin, erythromycin, amoxicillian, thimethoprimsulfamethoxale, doxycyclin, cefpodoxime, cefuroxime, cefdinir, clarithromycin, azithromycin, levofloxacin, gatifloxacin, and moxifloxacin.

Treatments may also involve administration of alpha-adreneric agonists such as oxymetazoline HCl, anticholinergic (parasympatholytic) agents such as ipratropium bromide, antihistamines such as chlorpheniramine maleate, beta-agonist bronchodilators, non-steroidal anti-inflammatory drugs, camphor, menthol, Echinacea, antiviral agents, mast cell stabilizers such as cromolyn sodium, topical nasal steroids such as fluticasone propionate and zinc salts.

In some embodiments, a particular therapeutic agent (e.g., a particular antibiotic) is selected for use in treatment in view of the results of the immunoassay. For example, the immunoassay can be used to identify a biomarker in the sinus fluid that is characteristic of a particular pathogen that is a causative agent of the sinusitis (e.g., a particular bacterial type and/or strain). A therapeutic agent (e.g., a particular antibiotic) that is effective to treat the particular pathogen identified by the immunoassay (e.g., a particular bacterial type and/or strain) can then be administered to the patient.

The devices, systems, and methods of the appended claims are not limited in scope by the specific devices, systems, kits, and methods described herein, which are intended as illustrations of a few aspects of the claims. Any devices, systems, and methods that are functionally equivalent are intended to fall within the scope of the claims. Various modifications of the devices, systems, kits, and methods in addition to those shown and described herein are intended to fall within the scope of the appended claims. Further, while only certain representative devices, systems, kits, and method method steps disclosed herein are specifically described, other combinations of the devices, systems, kits, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

1. A probe for assaying a biomarker in the sinus of a patient comprising
an elongate member having a proximal end and a distal end; and
a diagnostic element affixed to the distal end of the elongate member,
wherein the diagnostic element comprises an immunoassay for the presence of a biomarker that is characteristic of sinusitis.

2. The probe of claim 1, wherein the biomarker is associated with one or more causative agents of sinusitis.

3. The probe of claim 2, wherein the causative agent is selected from the group consisting of bacterial infections, viral infections, fungal infections, allergic reactions, and combinations thereof.

4. The probe of claim 1, wherein the biomarker is characteristic of bacterial sinusitis.

5. The probe of claim 4, wherein the biomarker is selected from the group consisting of outer membrane protein P5 (OMP P5), outer membrane protein P2 (OMP P2), or combinations thereof.

6. The probe of claim 1, wherein the diagnostic element comprises a lateral flow immunochromatographic assay.

7. The probe of claim 6, wherein the diagnostic element comprises a test liquid inlet for the lateral flow immunochromatographic assay at the distal end of the diagnostic element.

8. The probe of claim 1, wherein the biomarker is detected in sinus fluid located in the sinus of the patient.

9. The probe of claim 1, wherein a portion of the diagnostic element is positionable in the sinus of the patient using the elongate member.

10. The probe of claim 1, wherein a portion of the diagnostic element is positionable in contact with sinus fluid in the sinus of the patient using the elongate member.

11. The probe of claim 1, wherein the elongate member is flexible.

12. The probe of claim 1, further comprising a protective sheath that surrounds the diagnostic element.

13. The probe of claim 12, wherein the diagnostic element is retractably positioned within the protective sheath, such that actuation of the elongate member in the distal direction extends at least a portion of the diagnostic element beyond the distal end of the protective sheath.

14. The probe of claim 13, further comprising a membrane that seals the distal end of the protective sheath.

15. The probe of claim 14, wherein actuation of the elongate member in the distal direction extends at least a portion of the diagnostic element through the membrane.

16. The device of claim 1, wherein the elongate member is from about 5 cm to about 20 cm in length.

17-26. (canceled)

27. A system comprising
(i) the probe defined by claim 1; and
an endoscope comprising a lumen extending from the proximal end of the endoscope to (ii)
the distal end of the endoscope,
wherein the lumen is sized and configured such that the probe can be advanced through the lumen to expose a distal portion of the probe beyond the distal end of the endoscope.

28-37. (canceled)
